(19) United States
(12) Patent Application Publication
(10) Pub. No.: US 2005/0150502 A1
(43) Pub. Date: Jul. 14, 2005

(54) ABBREVIATED NONMOLDED ADHESIVE CONDOM

(76) Inventor: Valtann Ayres, Capital Heights, MD (US)

Correspondence Address: GREENBERG & LIEBERMAN, 314 PHILADELPHIA AVE., TAKOMA PARK, MD, 20912, US

(21) Appl. No.: 10/707,787

(22) Filed: Jan. 12, 2004

(51) Int. Cl. A61F 6/04

(52) U.S. Cl. 128/844

(57) ABSTRACT

An abbreviated condom that does not exceed the neckline of the penis. The present invention provides a secure contraceptive device, yet it still allows for the increased pleasure and the natural feeling for both people during intercourse. The present invention has an adhesive application that secures it around the head and neck of the penis, which prevents seepage of semen, it prevents the present invention from slipping off the penis during intercourse and it provides strong support to avoid breaking and tearing of the present invention. The tip of the present invention provides a narrow and closed end configuration with ample room on the closed end to collect seminal fluid. Following the tip, the present invention widens to fit over the glans of the penis. The present invention aids in the prevention of pregnancies and spread of STDs.

### BACKGROUND OF INVENTION

The present invention relates to an abbreviated condom device that diminishes the possibility of undesirable pregnancy and spread of sexually transmitted diseases, more particularly a non-molded abbreviated condom device that contains an adhesive material so that the device simply adheres to just the head of a penis, thus increasing user pleasure while preventing the leakage of semen.

Since its inception in the 18th century the conventional condom has been one of the oldest and most popular devices used for contraception and reducing the spread of sexually transmitted diseases (STDs). For the prevention of pregnancy, birth control pills, intrauterine devices (IUDs), diaphragms, and spermicidal jells, have been popular alternatives to condoms.

Yet, each of these condom alternatives is not without disadvantages For example, birth control pills, like many drugs, alter the body's natural chemistry. As a result, the use of the birth control pill is often accompanied by substantial side affects, such as irregular menstrual bleeding, nausea, weight gain, headaches, dizziness, breast tenderness, blood clots, and mood changes. IUDs are often correlated to serious medical problems and the insertion and presence of such devices cause many women discomfort. Also, the usage of diaphragms and spermicidal jells are inconvenient for many women because insertion of such devices are often complicated and such devices are required to be present for many hours after intercourse to be effective.

In light of such alternatives, the condom becomes even more valued because it is the only contraceptive method that is proven to not only prevent unwanted pregnancy but also prevent STDs. Presently the condom is the subject of medical and commercial interest, as the numbers of those infected worldwide with deadly STDs such as HIV and AIDS increase every year. The HIV and AIDS outbreak requires that those sexually active become educated on such diseases and consider protecting themselves from infection of HIV or an assortment of other STDs, such as chlamydia, herpes, syphilis, and gonorrhea. Also the increase of teenagers who are sexually active has caused extreme public interest in the condom in order to curtail unwanted pregnancies and STDs.

The conventional condom typically consists of an extended closed end casing, that when in use provides coverage on the penis head and shaft. Prior to application the casing is rolled down toward the closed end to form a disk configuration. A roll down method is typically employed for putting the condom in place on the penis shaft.

Despite the proven effectiveness of the conventional condom, many condoms are known to suffer drawbacks that cause them to be avoided by those sexually active. Consequently, exposing themselves to the risk of unwanted pregnancy and STDs. One of the drawbacks of conventional condoms is the element that the usage of a condom considerably interrupts the natural progression of sexual intercourse, more particularly the foreplay activity between participants immediate before sexual intercourse. In the prime of sexual arousal, the male must rush to open a condom package, remove the condom, and carefully and properly apply the condom to the penis. The condom, usually enclosed in lubricant fluid, is cold to the touch. Application of such a cold object to the penis is unpleasant to many individuals, and as a result discourages the use of the condom.

Other disadvantages of the conventional condom include the tendency for the condom to slip off the penis during usage, which causes the seepage of semen into the vaginal passage. Such slippage that results in seepage, defeats the purpose of using the condom in the first place.

The configuration of the conventional condom covers the most sensitive area of the penis, the coronal sulcu, thus as a result such a device causes a lack of desired sensitivity during sexual intercourse. Accordingly, many choose to avoid using condoms because of this loss of sensitivity during sexual intercourse that can result from usage. The introduction of ultra-thin condoms, attempts to curtail the loss of sensitivity in sexual intercourse caused by the usage of condoms. Yet these ultra thin varieties are more susceptible to seepage and tearing, thus they are not suitable for protection. Due to this, when designing conventional condoms it is optimal to use a thick material to eliminate the tendency of breakage, yet this will result in the reduction of user's sexual pleasure. This reduction in sexual pleasure consequently causes users to avoid using a condom even when failing to do so could have undesirable dangerous consequences.

As more information is learned about the tendencies of STDs, and as the numbers of unwanted pregnancies (such is the case in many teen pregnancies) rises, an assortment of abbreviated condoms have been formed in an attempt to address the loss of sexual pleasure while still providing sufficient protection.

U.S. Pat. No. 4,869,269 issued to Arnold L. Sharkan on Sep. 26, 1989 and U.S. Pat. No. 6,035,854 issued to Rory P. Blake on Mar. 14, 2000 show contraceptive-prophylactic devices that use adhesives to stick on the tip and glans of the penis. Unlike the present invention, these devices are made out of a thick material mold, meaning the condom is hardened and shaped around the glans and top of the penis. Such mold methods provide an unnatural feeling for the user during sexual intercourse.

U.S. Pat. No. 5,421,350 issued to Leah Friedman on Jun. 6, 1995 shows an unrolled condom that through adhesive means covers only the head of the penis. Unlike the present invention, this device does not extend to the neck of the penis, and even though this device has an adhesive means, it lack the full support and can easily slip off the penis head during intercourse.

U.S. Pat. No. 6,145,507 issued to Timothy J. Hardy on Nov. 14, 2000 shows an abbreviated condom that uses adhesive sticks to the tip of the glans of the penis, yet unlike the present invention this device releases spermicidal fluid and does not provide much room for semen flow, which can result in seepage. Also this device, contrasting to the present invention, does not exceed the lip of the penis.

U.S. Pat. No. 6,148,819 issued to John Andrew Winkler on Nov. 21, 2000 shows a method and kit containing a liquid adhesive for adhering a condom to the penis and a liquid solvent for removing the condom from the penis, yet unlike the present invention the condom does not come with the adhesive already on it. The user must preapply the adhesive coating, which interrupts the natural progression of sexual intercourse even more than a conventional condom. The device does not extend over the lip of the penis, as does the present invention. The device lacks sufficient space for the semen to collect after ejaculation. The device uses liquid adhesive, unlike the present invention, where the extension over the lip gives more security. The device is made out of a mold, which will provide an unnatural feeling for the users during sexual intercourse.

There exists a need for an abbreviated condom that provides the user a natural feeling, increases user pleasure, provides sufficient room for semen as a result prevents seepage, and provides strong support to avoid breakage of the condom, in addition to preventing pregnancy and spread of STDs.

### SUMMARY OF INVENTION

The present invention improves upon the various previous methods of contraception and STD prevention and cited patents by not covering the corona area, providing an optimal natural feel for the user, and including an adhesive configuration that is resistant to breakage and seepage of semen.

The present invention is comprised of an abbreviated condom that does not exceed the neckline of the penis. The present invention has an adhesive strip to adhere around the neck and head of the penis. The tip of the present invention is configured to suit the shape of the tip which is narrow and closed end. The present invention has a seminal reservoir for the collection of semen during ejaculation. The opposite end of the present invention widens out to fit over the glans of the penis.

The present invention is made of a non-molded thin, flexible, preferably a latex type material acts as a barrier to prevent the exchange of body fluids during sexual intercourse. The present invention's contour around the neck and head of the penis provides maximum, strength, support and comfort for the user. The thick band that rests along the penis neckline also provides support. This thick band is comprised of an elastic material with an adhesive strip attached. This adhesive strip is similar to the adhesive on adhesive bandages or breathing bands. The thick band in conjunction with the adhesive strip holds the condom in place by forming a leak proof seal with the penis neckline.

Because the present invention features this adhesive strip to secure it, there is no need for the user to employ the roll down method employed when using a conventional condom. The present invention does not cover the most sensitive area of the penis, which means that there is no primary drawback and no decrease in the pleasure of sexual intercourse. The present invention's configuration envelops only the head and neckline of the penis, leaving the sensitive corona of the penis free, thus enhancing user pleasure.

The present invention is made of a thin flexible material, while the thick band is twice the thickness of a regular condom. In addition, the adhesive is not located on the glans nor lip of the penis, instead the adhesive is exclusively located on the thick band around the neckline of the penis. Such a configuration provides a natural feel for the user, yet at the same time provides protection from semen escaping from the seminal reservoir.

The user gently slides the present invention onto the head of the penis. The thick part is then pulled over the head, on to the neckline of the penis. Afterward, the user pulls the adhesive transfer film from the thick band of the present invention in order to expose the adhesive. Pressing the thick band, containing adhesive, around all sides of the penis ensures added support as well as a natural feel for the user.

### BRIEF DESCRIPTION OF DRAWINGS

FIG. 1 is a side view of the present invention in use before the application of the adhesive strip.

FIG. 2 is a top view of the present invention in use with the application of the adhesive strip.

FIG. 3 is a bottom view of the present invention in use with the application of the adhesive strip.

FIG. 4 is a back view of the present invention such that that the open end of the present invention can be seen, showing the adhesive tab before it is removed.

FIG. 5 is a top, side, and bottom view of the present invention showing the adhesive band flipped over and exposed after the tab has been removed.

### DETAILED DESCRIPTION

The present invention is an abbreviated condom comprised of a tapered closed end seminal reservoir 10, a thick elastic band 30 concluding at the neckline of the penis, that contains an adhesive strip 40 with transfer film 50 on the under side and a concluding widened openended portion 20.

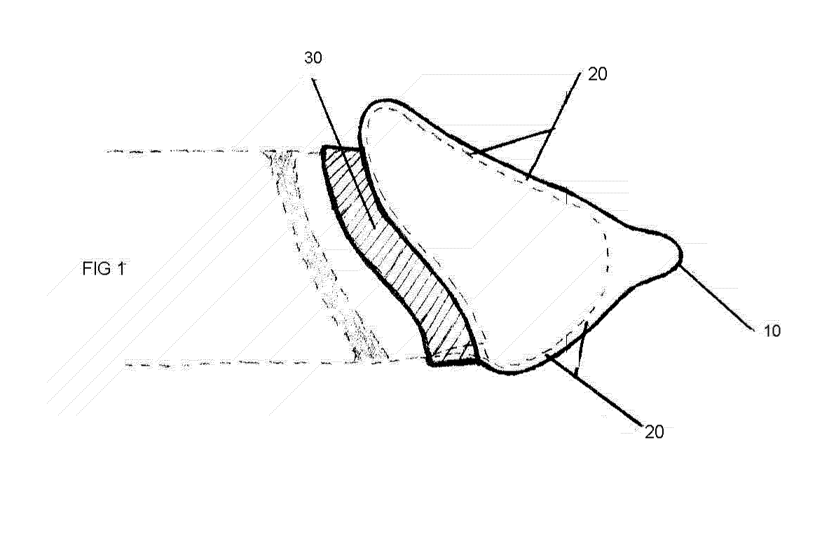

Turning to FIG. 1, the tapered closed end seminal reservoir 10 and widened open-ended portion 20 of the present invention are made of a non-molded, thin, flexible material, preferably latex, rubber, plastic or polyurethane. Such material is sufficiently resilient enough to prevent rupturing or tearing of the present invention, which leads to semen leakage, during placing of the present invention on the penis and during sexual intercourse. Yet such material is also thin enough to provide a natural feeling for both parties during sexual intercourse. The contour fit of the thick elastic band 30 with the underpinning of the adhesive strip 40 causes the present invention to fit snuggly and securely around the head and neckline of the penis. The seminal reservoir 10 provides ample room to catch the seminal fluid during ejaculation.

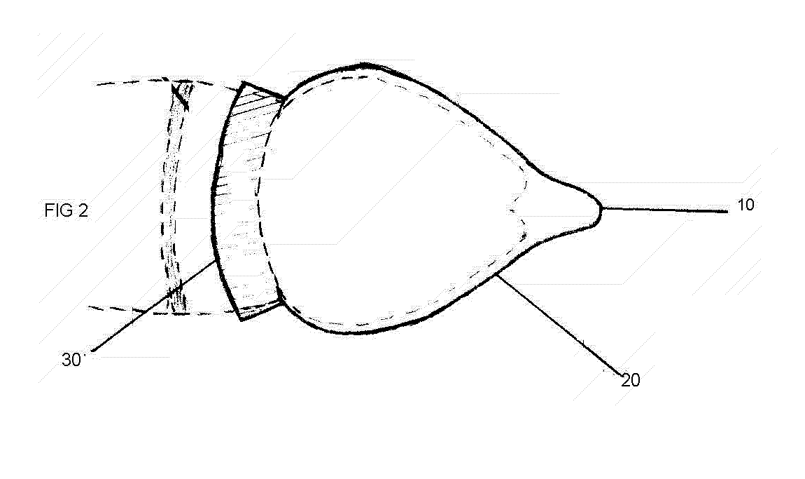

Turning to FIG. 2, the thick elastic band 30, which does not exceed the penis neckline, is formed of an elastic material that is twice the thickness of a conventional condom. The thick elastic band 30 stops at the neck of the penis. Underneath the thick elastic band 30 is the adhesive strip 40 that adheres the present invention to the penis. The elastic thick band 30 is a prime importance in the present invention because it provides for a leak-proof seal to prevent semen leakage, yet also allows the user a natural feel while wearing the present invention. It is important that the elastic thick band 30 rests at the neckline of the penis because this ensures the best protection against leakage.

Semen is stored in the lops of the penis. The present invention is specifically designed to provide adhesive around the underside of the thick elastic band 30 to ensure that no semen leaks out of the present invention; if it were not designed this way there is a good probability that leakage of semen will occur. To use the present invention, the user gently slides the present invention onto the head of an erected penis. The elastic thick band 30 is then pulled over the head, onto the neckline of the penis and secures it by removing the transfer film 50 from underneath the thick elastic band 30 of the present invention in order to expose the adhesive strip 40.

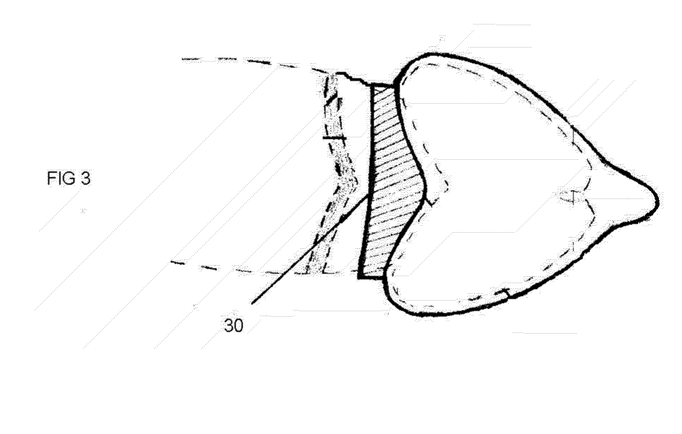

Turning to FIG. 3, this is a bottom view of the penis with the present invention detailing that the thick elastic band 30 does not exceed the neckline of the penis and that the wide open-ended portion 20 contours under the two lops of the penis. FIG. 3 emphasizes the secure fit of the present invention around all sides of the penis.

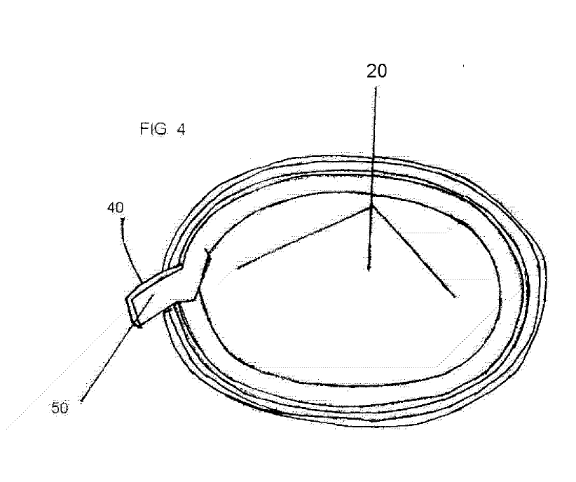

Turning to FIG. 4, the layer of adhesive strip 40 is shown with the transfer film 50 still attached. The adhesive strip 40 is similar to the adhesive used in adhesive bandages or breathing strips. The user pulls the present invention onto his penis, the transfer film 50 is peeled away; thus exposing the adhesive strip 40 of the thick elastic band 30, is exposed and pressed in place around all sides of the penis. This adhesive strip 40 is located only around the neckline of the penis, permitting the user a natural feel, instead of causing the user to feel extremely uncomfortable due to an enormous amount of adhesive on the skin. Prior to fitting of the present invention, the transfer film 50 is peeled away to expose the adhesive strip 40. When the adhesive coating 40 is exposed and applied, the adhesive strip 40 prevents leakage by forming a leak proof seal along the neckline of the penis. This is done prior to sexual intercourse. Once the sexual encounter is over, the user can remove the present invention, by simply peeling the present invention from around the neck of the penis. Then the user can dispose of the used present invention.

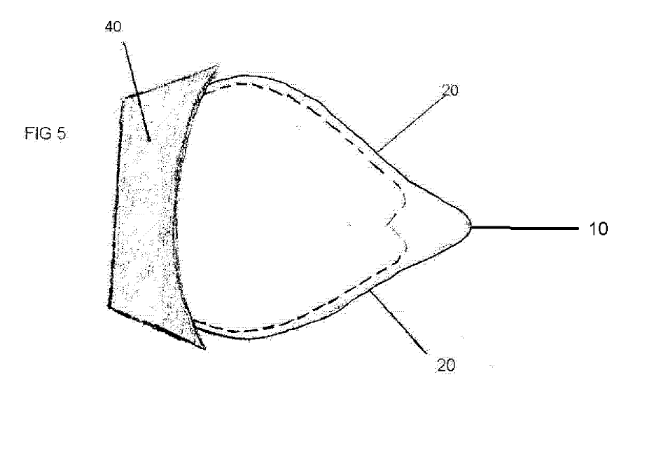

Turning to FIG. 5, the present invention is shown with the adhesive strip 40 exposed showing after the transfer film 50 has been removed. This view shows the extinct of the area that is coated with the adhesive strip 40. It shows that the underside of the thick elastic band 30 is well supplied with the adhesive strip 40 to ensure a snug, secure and tight seal for the present invention around the neck of the penis.

The abbreviated length of material that the present invention represents, in comparison to a conventional condom, is short enough to fit the head of a penis, yet also allow for ample protection from leakage and increase pleasure for both participants in the sexual encounter. The present invention contours only about the neck and head of the penis, and leaves the sensitive corona of the penis fully exposed. Thus the problem associated with conventional condoms of reduced user pleasure is solved through usage of the present invention. The special configuration of the present invention also permits for the elastic thick band 30 of the present invention to be made of a thick material, in order to prevent breakage and diminished pleasure for the user. Due to the adhesive strip 40 located on the elastic thick band 30, the tendency of leakage problems is removed by forming a complete semen leak proof seal at the neckline of the penis. Also the location of the adhesive strip 40 allows the remainder of the present invention to be made of a light flexible material, which will provide an optimal natural feel for the user, instead of a molded hardened material. One of the best benefits that the present invention has when compared to conventional condoms is that the present invention, through the use of adhesive strip 40 along the penis neckline, provides the user an optimal natural feel along with maximum protection from semen seepage.

1. A device for sexual intercourse, comprising:
a main body; and
a seminal reservoir, in combination with said main body.

2. The device of claim 1, wherein said main body is of a thin material.

3. The device of claim 1, wherein said main body is non-molded.

4. The device of claim 1, wherein said main body is flexible.

5. The device of claim 1, wherein said main body is made of latex, rubber, plastic or polyurethane.

6. The device of claim 1, wherein said seminal reservoir is of a thin material.

7. The device of claim 1, wherein said seminal reservoir is non-molded.

8. The device of claim 1, wherein said seminal reservoir is flexible.

9. The device of claim 1, wherein said seminal reservoir is made of latex, rubber, plastic or polyurethane.

10. The device of claim 1, further comprising a securing means in communication with said main body.

11. The device of claim 10, wherein said securing means is an elastic band.

12. The device of claim 10, wherein said securing means is an adhesive strip.

13. The device of claim 10, wherein said securing means is an elastic band in combination with an adhesive strip.

14. The device of claim 12, further comprising transfer film in communication with said adhesive strip.

15. The device of claim 13, further comprising transfer film in communication with said adhesive strip.

16. The device of claim 1, wherein said main body is wide.

17. The device of claim 1, wherein said seminal reservoir tapers.
